(19) United States
(12) Patent Application Publication
(10) Pub. No.: US 2025/0197355 A1
(43) Pub. Date: Jun. 19, 2025

(54) TEAD Targeting Compounds and Methods Thereof

(71) Applicant: Agency for Science, Technology and Research, Singapore (SG)

(72) Inventors: Joel Dewei Toh, Proteos (SG); Wanjin Hong, Proteos (SG); Ramesh Kumar, Proteos (SG)

(73) Assignee: Agency for Science, Technology and Research, Singapore (SG)

(21) Appl. No.: 18/867,750

(22) PCT Filed: Mar. 28, 2023
(86) PCT No.: PCT/SG2023/050202

§ 371 Date: Nov. 20, 2024

(30) Foreign Application Priority Data
May 20, 2022 (SG) 10202205354V

(51) Int. Cl. C07D 213/75; A61K 31/415; A61K 31/4192; A61K 31/426; A61K 31/44; A61K 45/06; C07D 231/40; C07D 249/06; C07D 277/46

(52) U.S. Cl. CPC C07D 213/75; A61K 31/415; A61K 31/4192; A61K 31/426; A61K 31/44; A61K 45/06; C07D 231/40; C07D 249/06; C07D 277/46

(57) ABSTRACT

The present invention relates, in general terms, to TEAD targeting compounds and 5 methods thereof.

### TECHNICAL FIELD

The present invention relates, in general terms, to TEAD targeting compounds and methods thereof.

### BACKGROUND

The Hippo pathway plays a central role in regulating organ size and maintaining dynamic tissue balance. The pathway involves TAOK1/2/3 phosphorylation or MST1/2 automatic phosphorylation which initiates the Hippo kinase cascade. MST1/2 activation phosphorylates LATS1/2. The activated LATS1/2 phosphorylates YAP/TAZ under the action of SAV1, MOB1A/B, and NF2. This results in the 14-3-3-mediated cytoplasmic retention and SCF-mediated degradation of YAP/TAZ. YAP/TAZ is a transcriptional coactivator that regulates gene transcription mainly by interacting with TEAD. The upregulation of TEAD target gene expression, with partial deletion of kinase cascade or YAP overexpression, can lead to increased progenitor cell proliferation and tissue overgrowth.

Because of the Hippo pathway's unique ability to promote regeneration, any abnormality of its core components, may promote the migration, invasion, and malignancy of cancer cells. Aberrant overexpression of YAP/TAZ in tumors promotes tumorigenesis and is therefore considered an oncogene in a large number of solid cancers. Drug resistance is a major factor undermining the efficacy of cancer drugs. As YAP/TAZ-TEAD plays a role in intrinsic and acquired resistance to various chemotherapeutic and targeted therapy drugs, there is increasing interest in combining a TEAD inhibitor with various cancer therapy.

Growing body of evidence strongly suggest that elevated YAP/TAZ-TEAD activity has been implicated in multiple stages of cancer progression and cancer types. Drugging the TEAD hydrophobic pocket, which was discovered in 2015, remained an attractive and proven strategy to modulate the activity. Despite the importance, only 3 small molecule TEAD inhibitors are currently being tested in Phase I clinical trials which includes: VT3989b NCT04665206 from Vivacare, IK-930b NCT05228015 from Ikena Oncology and IAG933b NCT0485737 from the Novartis Oncology. As such, there is a need to uncover alternative TEAD inhibitors for cancer therapeutics.

It would be desirable to overcome or ameliorate at least one of the above-described problems.

### SUMMARY

The present disclosure is predicated on the understanding that TEAD inhibitors may have enormous therapeutic potential if they are administered to patients whose tumors express the YAP/TAZ-TEAD signature, as they are very likely to respond to TEAD inhibitor therapy.

The present disclosure concerns covalent chemical scaffolds which may hijack the conserved cysteine of the TEAD. For example, CPD10 and CPD13 resulted in down regulation of TEAD regulated transcriptional target genes. Immunoprecipitation and Immunoblotting data indicate that CPD10 and CPD13 resulted in disruption of TEAD4 binding to YAP/TAZ and reduces the expression of target proteins in a dose dependent manner. Cellular proliferation and colony formation assay indicate that CPD10 and CPD13 resulted in inhibition of cell growth and viability in Osteosarcoma (U20S) and Lung cancer cells (A549).

The present disclosure concerns a compound of Formula (I) or a salt, solvate or prodrug thereof:

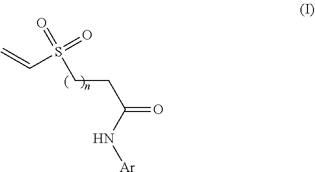

wherein
n is an integer selected from 1 to 5; and
Ar is an optionally substituted aryl or optionally substituted heteroaryl.

In some embodiments, Ar is optionally substituted heteroaryl.

In some embodiments, Ar is an optionally substituted heteroaryl, wherein a heteroatom is at a 2′ position relative to N of the amide moiety.

In some embodiments, Ar is selected from optionally substituted phenyl, optionally substituted pyridinyl, optionally substituted thiazolyl, optionally substituted pyrazolyl, or optionally substituted triazolyl.

In some embodiments, n is an integer selected from 1 to 4.

In some embodiments, the compound of Formula (I) is a compound of Formula (Ia):

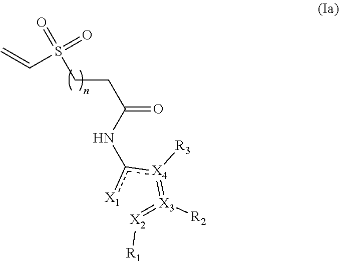

wherein
n is an integer selected from 1 to 5;
X₁ is a heteroatom selected from N, S, or O;
X₂, X₃ and X₄ are independently selected from C, N, O, or S;
when X₂ is N or C, R₁ is selected from optionally substituted cycloalkyl, optionally substituted heterocyclyl, optionally substituted aryl, or optionally substituted heteroaryls;
when X₃ and X₄ are independently N or C, R₂ and R₃ are independently selected from H, or optionally substituted alkyl.

In some embodiments, X₁ is a heteroatom selected from N, or S.

In some embodiments, at least one of X₂, X₃ and X₄ is N, O, or S.

In some embodiments, R₁ is selected from optionally substituted cycloalkyl or optionally substituted aryl.

In some embodiments, at least one of R₂ and R₃ is optionally substituted alkyl.

In some embodiments, the compound of Formula (I) is selected from:

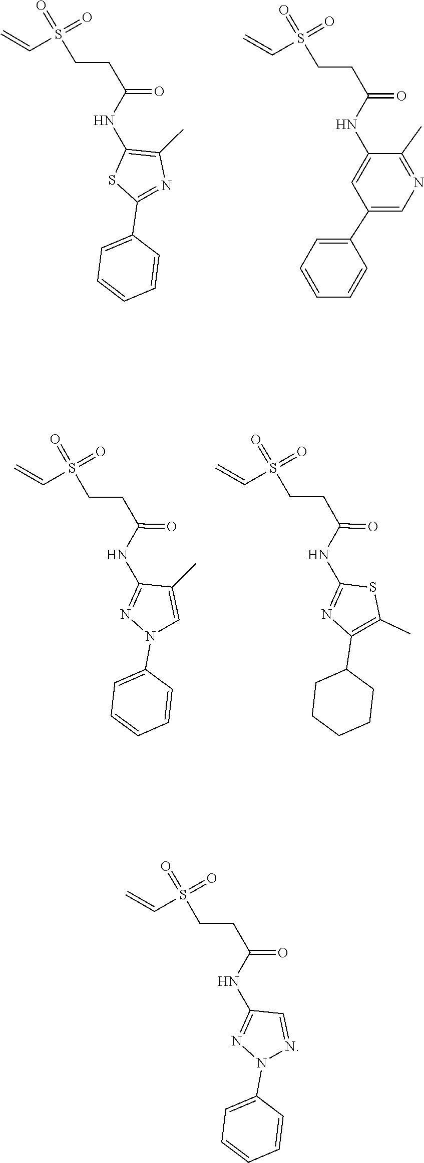 [SMILES: C=CS(=O)(=O)CCC(=O)Nc1sc(-c2ccccc2)nc1C ; C=CS(=O)(=O)CCC(=O)Nc1cc(-c2ccccc2)cnc1C ; C=CS(=O)(=O)CCC(=O)Nc1nn(-c2ccccc2)cc1C ; C=CS(=O)(=O)CCC(=O)Nc1nc(C2CCCCC2)c(C)s1 ; C=CS(=O)(=O)CCC(=O)Nc1cnn(-c2ccccc2)n1]

The present disclosure concerns a modulator of Hippo pathway, wherein the modulator is a compound of Formula (I). The compound of Formula (I) is a modulator of YAP/TAZ-TEAD.

The present disclosure also concerns a pharmaceutical composition comprising an effective amount of a compound of Formula (I) or a pharmaceutically acceptable salt, solvate or prodrug thereof, optionally in combination with a pharmaceutically acceptable carrier, excipient or diluent.

The present disclosure also concerns a method of treating cancer in a patient in need thereof, comprising administering to the patient a therapeutically effective amount of a compound of Formula (I) or a pharmaceutically acceptable salt, solvate or prodrug thereof.

The present disclosure also concerns a compound of Formula (I) or a pharmaceutically acceptable salt, solvate or prodrug thereof for use in the treatment of cancer.

The present disclosure also concerns a use of a compound of Formula (I) or a pharmaceutically acceptable salt, solvate or prodrug thereof in the manufacture of a medicament for treating cancer in patient in need thereof.

In some embodiments, the cancer is characterised by an elevated YAP/TAZ-TEAD activity and/or drug resistance.

In some embodiments, the cancer is selected from mesothelioma, liver cancer, gastric cancer, metastatic non-small-cell lung cancer (NSCLC) and colorectal cancer.

### BRIEF DESCRIPTION OF THE DRAWINGS

Embodiments of the present invention will now be described, by way of non-limiting example, with reference to the drawings in which:

FIG. 1. A) Confirmation of palmitate and reported inhibitors adopted in TEAD pocket. B) Superimposition of palmitate and reported inhibitors revealed similar physical and chemical properties (hydrogen donor and acceptors) that facilitated potent and selective binding to TEAD pocket. Position of cysteine is primed to be leveraged. C) Examples of three different cysteine warheads selected for screening.

FIG. 2. A) Schematic of activity-based protein profiling (ABPP). Covalent compound binding to conserved cysteine reduces the fluorescence signal. If the conserved cysteine is not labelled, there will be more fluorescence. B) Palmitoyl Coenzyme A used a positive control to show that we have optimized ABPP for our assay. C) Screening campaign with arbitrary cut-off of 0.25. Compounds with less than 0.25 normalized fluorescence was considered as hit. (N=4 independent experiments). D) Typical gel readout for screening campaign; Palmitoyl Coenzyme A as a positive control; DMSO as negative control; Compounds activities were normalized by: Setting DMSO as 1 and Palmitoyl Coenzyme A as 0.

FIG. 3. A) Intact mass spectrometry (MALDI) showed labeling of TEAD by compounds (mass peak shifts right correspond to one compound) in vitro. Notably, only one out of the four cysteine residues react with the covalent compounds. Not all the compounds reacted with TEAD, reflecting the cysteine warhead do not indiscriminately label any cysteine residue. B) SAR of the hits. C) LC-MS/MS assay revealed the more preferred cysteine residue labeled is the conserved cysteine.

FIG. 4. A) CPD10 and CPD13 inhibits the expression of YAP/TAZ proteins: A549 cells treated with indicated doses of CPD10 and immunoblotted for YAP, YAP/TAZ, pan-TEAD. GAPDH and p53 signals were used a control (A). Likewise, A549 cell treated with cpd13 reduced YAP protein signal at 10 μM (B). In response to 10 μM of CPD10 treatment, YAP/TAZ binding of TEAD4 was significantly reduced (C).

FIG. 5. CPD10 and CPD13 promoted Cytoplasmic localization of TEAD4: CPD10 and CPD13 treatment resulted in TEAD4 cytoplasmic localization signal: Unlike DMSO treated cells CPD10 and CPD13 resulted in cytoplasmic signal while nuclear signal is still intact.

FIG. 6. CPD10 and CPD13 resulted in downregulation of TEADs transcriptional targets: Relative expression of YAP and TAZ in response to low dose of CPD10 (A) and CPD13 (B) treatment in A549 cells. c-MYC was included as non-transcriptional target of the TEADs. NFKB1 was included as its expression is reciprocal to the YAP/TAZ. Downstream effectors of the TEADs targets (CYR61, AXL, CTGF and ANKRD1) were determined in responses to 2.5, 5 and 10 μM of CPD10 (C) and CPD13 (D) respectively.

FIG. 7. CPD10 and CPD13 displayed TEAD4 dependent cellular proliferation and differential viability in U2OS and A549 cells: Cellular proliferation assay data indicating that CPD10 strongly sensitizing TEAD4 overexpressing U20S cells and A549 cells (A and B). Depletion of TEAD4 in A549 cells resulted in reduction of CPD10 induced cellular proliferation (C). Colony forming assay indicating that compared to GFP overexpressing cells, GFP-TEAD4 cells are more sensitive to the 2.5 μM of CPD10. Unlike control siRNA treated cells, TEAD4 depleted cells are less sensitive to CPD13 (D).

FIG. 8. TEAD modelling and evaluation of CPD10 and CPD13 in NCI-H226 mesothelioma cell line: In-silico docking for modeling TEAD-CPD10/CPD13 (A and B). Modeling TEAD-CPD10 (C). TEAD4 immunoprecipitation in NCI-H226 mesothelioma cells treated with indicated concentrations of CPD10 and CPD13 to determine YAP-TEAD binding (D). Immunoblot gel quantification to determine the % of YAP-TEAD binding disruption (E). YAP-TEAD transcriptional target genes profiling (F). Dose dependent relative luminescence (ATP/ADP ration) profiling in responses to CPD10 and CPD13 treated NCI-H226 mesothelioma cells (G). Colony forming assay to determine the CPD10 and CPD 13 induced drug sensitivity and ability to form colonies in NCI-H226 mesothelioma cells (H).

FIG. 9 compares CPD13 with a positive control in different cells.

FIG. 10 shows colony forming assay to determine cell viability in response to CPD2 treatment and recovery (5 days).

FIG. 11 shows colony forming assay to determine cell viability in response to CPD38 treatment and recovery (5 days).

### DETAILED DESCRIPTION

“Alkyl” refers to monovalent alkyl groups which may be straight chained or branched and preferably have from 1 to 10 carbon atoms or more preferably 1 to 6 carbon atoms. Examples of such alkyl groups include methyl, ethyl, n-propyl, iso-propyl, n-butyl, iso-butyl, n-hexyl, and the like.

“Alkenyl” refers to a monovalent alkenyl group which may be straight chained or branched and preferably have from 2 to 10 carbon atoms and more preferably 2 to 6 carbon atoms and have at least 1 and preferably from 1-2, carbon to carbon, double bonds. Examples include ethenyl (—CH═CH₂), n-propenyl (—CH₂CH═CH₂), iso-propenyl (—C(CH₃)=CH₂), but-2-enyl (—CH₂CH═CHCH₃), and the like.

“Halo” or “halogen” refers to fluoro, chloro, bromo and iodo.

“Oxo/hydroxy” refers to groups ═O, HO—.

“Aryl” refers to an unsaturated aromatic carbocyclic group having a single ring (eg. phenyl) or multiple condensed rings (eg. naphthyl or anthryl), preferably having from 6 to 14 carbon atoms. Examples of aryl groups include phenyl, naphthyl and the like.

“Heteroaryl” refers to a monovalent aromatic heterocyclic group which fulfils the Hückel criteria for aromaticity (ie. contains 4n+2 n electrons) and preferably has from 2 to 10 carbon atoms and 1 to 4 heteroatoms selected from oxygen, nitrogen, selenium, and sulfur within the ring (and includes oxides of sulfur, selenium and nitrogen). Such heteroaryl groups can have a single ring (eg. pyridyl, pyrrolyl or N-oxides thereof or furyl) or multiple condensed rings (eg. indolizinyl, benzoimidazolyl, coumarinyl, quinolinyl, isoquinolinyl or benzothienyl).

Examples of heteroaryl groups include, but are not limited to, oxazole, pyrrole, imidazole, pyrazole, pyridine, pyrazine, pyrimidine, pyridazine, indolizine, isoindole, indole, indazole, purine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, thiazole, thiadiazoles, oxadiazole, oxatriazole, tetrazole, thiophene, benzo[b]thiophene, triazole, imidazopyridine and the like.

“Heterocyclyl” refers to a monovalent saturated or unsaturated group having a single ring or multiple condensed rings, preferably from 1 to 8 carbon atoms and from 1 to 4 hetero atoms selected from nitrogen, sulfur, oxygen, selenium or phosphorous within the ring. The most preferred heteroatom is nitrogen. It will be understood that where, for instance, R₂ or R′ is an optionally substituted heterocyclyl which has one or more ring heteroatoms, the heterocyclyl group can be connected to the core molecule of the compounds of the present invention, through a C—C or C-heteroatom bond, in particular a C—N bond.

Examples of heterocyclyl and heteroaryl groups include, but are not limited to, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiadiazoles, oxadiazole, oxatriazole, tetrazole, thiazolidine, thiophene, benzo[b]thiophene, morpholino, piperidinyl, pyrrolidine, tetrahydrofuranyl, triazole, and the like.

“Amino” refers to the group —NR″R″ where each R″ is independently hydrogen, alkyl, cycloalkyl, aryl, heteroaryl, and heterocyclyl and where each of alkyl, cycloalkyl, aryl, heteroaryl and heterocyclyl is as described herein.

In this specification “optionally substituted” is taken to mean that a group may or may not be further substituted or fused (so as to form a condensed polycyclic group) with one or more groups selected from hydroxyl, acyl, alkyl, alkoxy, alkenyl, alkenyloxy, alkynyl, alkynyloxy, amino, aminoacyl, thio, arylalkyl, arylalkoxy, aryl, aryloxy, carboxyl, acylamino, cyano, halogen, nitro, phosphono, sulfo, phosphorylamino, phosphinyl, heteroaryl, heteroarylalkyl, heteroaryloxy, heterocyclyl, heterocyclylalkyl, heterocyclyloxy, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-alkylamino, mono- and di-(substituted alkyl)amino, mono- and di-arylamino, mono- and di-heteroarylamino, mono- and di-heterocyclyl amino, and unsymmetric di-substituted amines having different substituents selected from alkyl, aryl, heteroaryl and heterocyclyl, and the like, and may also include a bond to a solid support material, (for example, substituted onto a polymer resin). For instance, an “optionally substituted amino” group may include amino acid and peptide residues.

The genetic alteration of Hippo pathway components has been increasingly detected in cancers. In this regard, small molecule covalent inhibitors of similar scaffold which can enter the TEAD pocket may be therapeutically useful against cancers associated with elevated activity of YAP/TAZ-TEAD relative to norm.

An alternative strategy involves identifying scaffolds that tightly bind to the hydrophobic pocket. For example, covalent TEAD inhibitors may be developed to leverage the conserved cysteine. Without wanting to be bound by theory, it is believed that the central hydrophobic pocket of TEADs may be targeted more specifically by combining these two strategies to identify scaffolds that simultaneously occupy the pocket, and binds covalently to the conserved cysteine, with the aim to achieve high potency and selectivity.

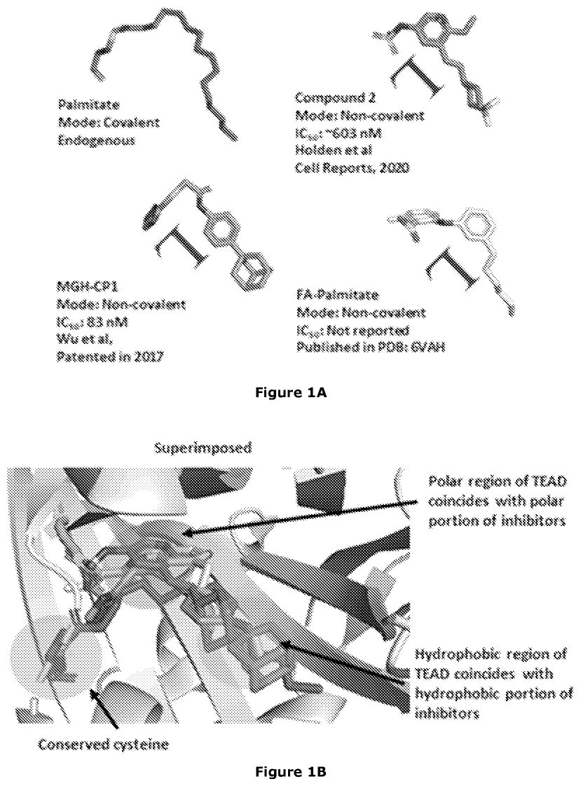

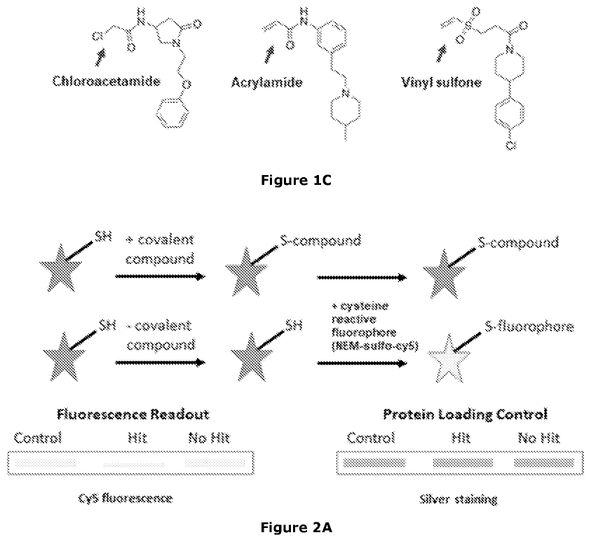

It was found that palmitoylation of the TEAD conserved cysteine is needed for its stability and activity. Here, compounds with a chemical scaffold that preferentially and covalently bind to the conserved cysteine in the palmitate binding pocket of TEAD were identified. A rational approach for compound library selection was employed. The available co-crystal structures of TEAD with palmitate and other non-covalent inhibitors were analysed and physical and chemical similarities were identified which guided the rational selection of new compound class for screening (FIGS. 1 A & B).

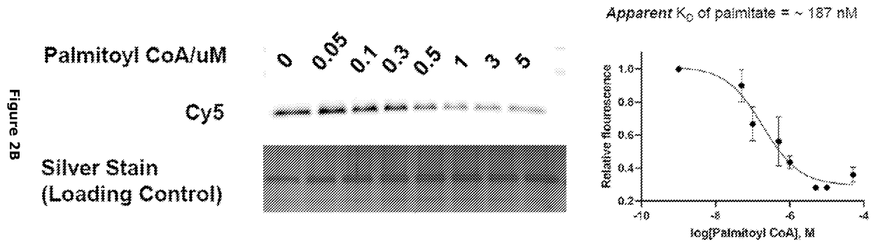

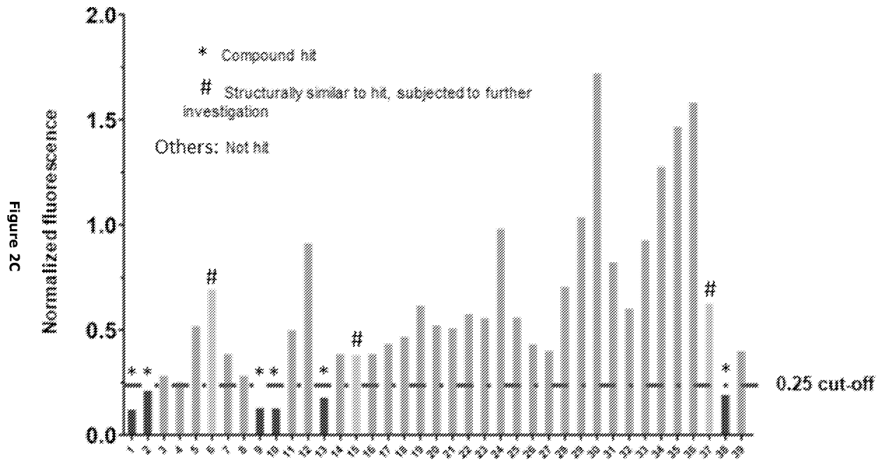

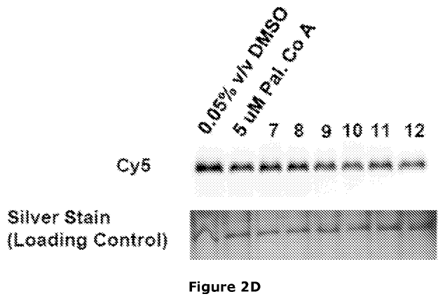

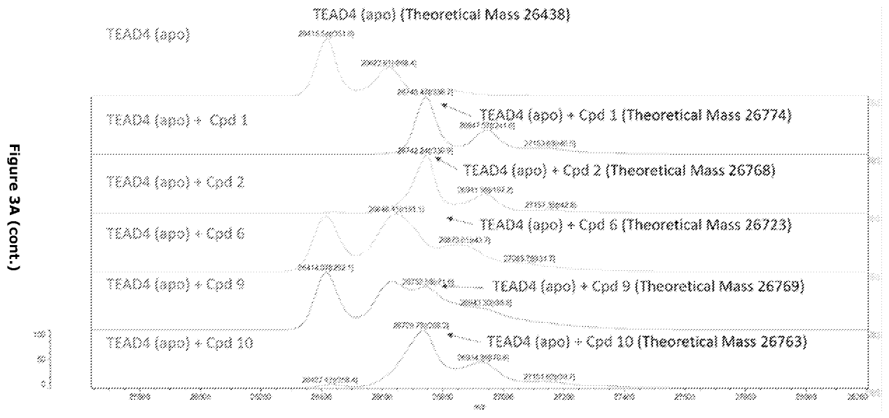

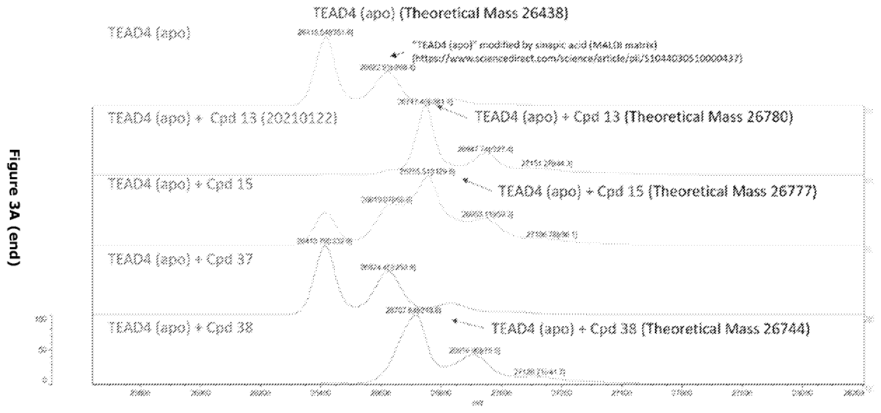

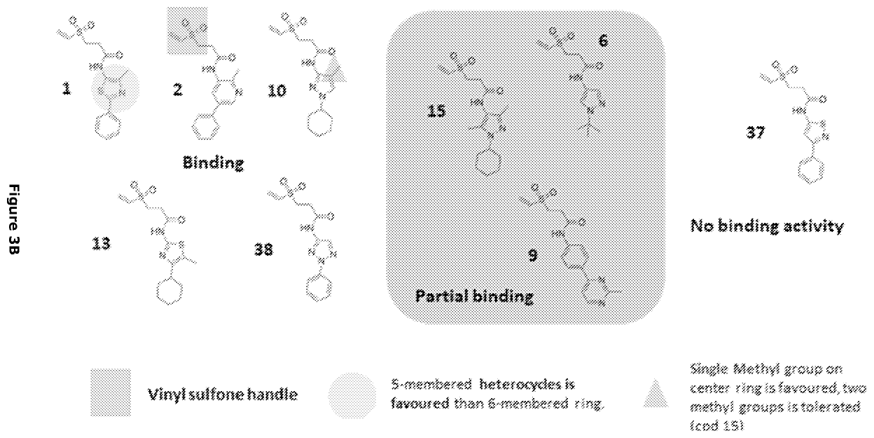

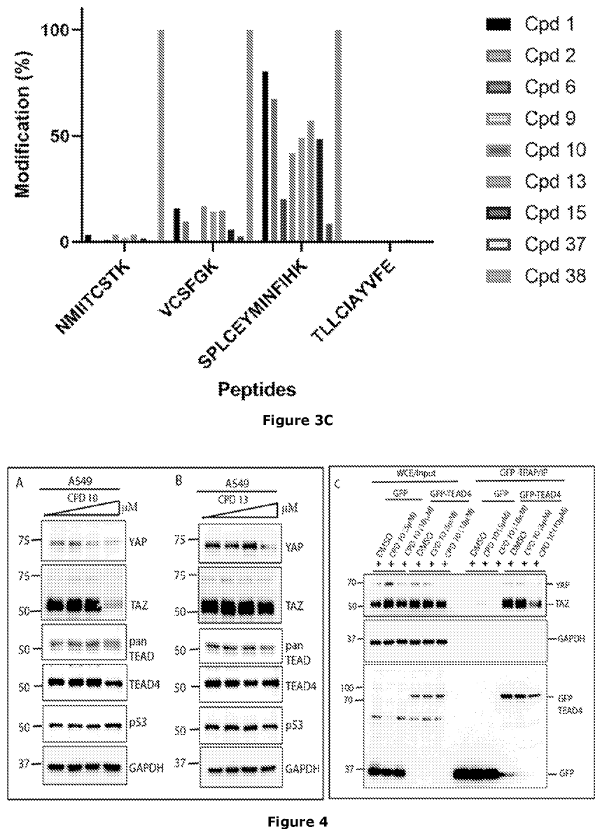

The rationally selected compound library pool comprises a cysteine warhead, preferably selected from chloroacetamide, acrylamide and vinyl sulfone (FIG. 1C). Next, a fluorescence gel-based activity-based protein profiling (ABPP) assay (FIGS. 2A & B) was designed to screen our library for covalent compounds that bind to the conserved cysteine of TEAD (FIG. 2C). The best compounds were counter screened to verify the binding activity using an intact mass-spectrometry assay (FIG. 3A), and at the same time, obtained useful information on the structure-activity relationship (SAR) (FIG. 3B). Further, LC-MS/MS data showed that the preferred site of modification is the conserved cysteine residue (FIG. 3C), where labelling prevented TEAD palmitoylation. Interestingly, vinyl sulfone warhead are particularly efficient. Our cell based and biochemical assay data strongly suggests a potential application in cancer therapeutics.

Accordingly, the present disclosure provides a compound of Formula (I) or a salt, solvate or prodrug thereof:

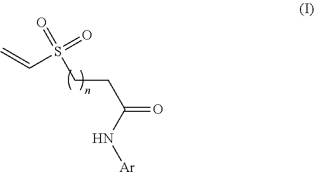

wherein
n is an integer selected from 1 to 5; and
Ar is an optionally substituted aryl or optionally substituted heteroaryl.

These compounds are rationally selected from Enamine, which were not previously known to have any biological property. These compounds demonstrate TEAD inhibition properties and may be developed for cancer therapeutics.

These chemical scaffolds (for example CPD10 and CPD13) have a cysteine warhead (vinyl sulfone) that targets TEAD cysteine. Further, in addition to targeting the hydrophobic pocket of TEAD, the compounds are optimised to interact with the polar residues near the entrance of the pocket. The present invention is applicable in cancer therapeutic for elevated YAP/TAZ-TEAD activities cancers and drug resistance. Multiple studies underline the significance of TEADs in human cancers. Overexpression of TEADs has been implicated in multiple stages of cancer progression and cancer types. Additionally, the Hippo pathway is known to be key factor developing resistance to chemo- and targeted therapies including Ras, EGFR, RAF and MEK pathway inhibitors.

In some embodiments, Ar is optionally substituted heteroaryl. In some embodiments, Ar is selected from optionally substituted phenyl, optionally substituted pyridinyl, optionally substituted thiazolyl, optionally substituted pyrazolyl, or optionally substituted triazolyl.

In some embodiments, the optional substituent is selected from optionally substituted aryl, optionally substituted cycloalkyl, optionally substituted heterocyclyl, and optionally substituted heteroaryl. In some embodiments, the optional substituent is selected from optionally substituted phenyl and optionally substituted cyclohexyl. In some embodiments, the optional substituent is selected from phenyl and cyclohexyl.

In some embodiments, the optional substituent is selected from halo, oxo, optionally substituted alkyl, optionally substituted alkoxy, optionally substituted alkyenyl and optionally substituted amino. In some embodiments, the optional substituent is selected from optionally substituted C₁-C₈alkyl, optionally substituted C₁-C₈alkoxy and optionally substituted C₂-C₅ alkyenyl. In some embodiments, the optional substituent is selected from C₁-C₈alkyl, C₁-C₅ alkoxy and C₂-C₅ alkyenyl.

In some embodiments, Ar is an optionally substituted heteroaryl, wherein a heteroatom is at a 2′ position relative to N of the amide moiety.

In some embodiments, n is an integer selected from 2 to 5, 3 to 5, or 4 to 5. In some embodiments, n is an integer selected from 1 to 4, 1 to 3, or 1 to 2. In some embodiments, n is 1.

In some embodiments, Ar is an optionally substituted heteroaryl. In some embodiments, Ar is an optionally substituted 5 membered heteroaryl. In some embodiments, the compound of Formula (I) is a compound of Formula (Ia):

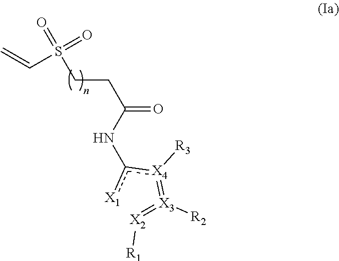

wherein
n is an integer selected from 1 to 5;
X₁ is a heteroatom selected from N, S, or O;
X₂, X₃ and X₄ are independently selected from C, N, O, or S;
R₁ is selected from optionally substituted cycloalkyl, optionally substituted heterocyclyl, optionally substituted aryl, or optionally substituted heteroaryls;
R₂ and R₃ are independently selected from H, or optionally substituted alkyl.

In some embodiments, the compound of Formula (I) is a compound of Formula (Ia):

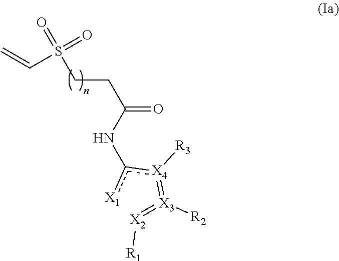

wherein
n is an integer selected from 1 to 5;
X₁ is a heteroatom selected from N, S, or O;
X₂, X₃ and X₄ are independently selected from C, N, O, or S;
when X₂ is N or C, R₁ is selected from optionally substituted cycloalkyl, optionally substituted heterocyclyl, optionally substituted aryl, or optionally substituted heteroaryls;
when X₃ and X₄ are independently N or C, R₂ and R₃ are independently selected from H, or optionally substituted alkyl.

Based on SAR studies, it was found that it can be desirable for X₁ to be a heteroatom. It is believed that a heteroatom at X₁ may interact beneficially with the TEAD hydrophobic pocket, presumably with peptide backbone or some proximal polar amino acid.

In some embodiments, X₁ is a heteroatom selected from N, or S. In some embodiments, X₁ is N. In some embodiments, X₁ is S.

In some embodiments, at least one of X₂, X₃ and X₄ is N, O, or S. In some embodiments, at least one of X₂, X₃ and X₄ is N or S.

In some embodiments, at least two of X₂, X₃ and X₄ is N, O, or S. In some embodiments, at least two of X₂, X₃ and X₄ is N or S.

In some embodiments, R₁ is selected from optionally substituted aryl, optionally substituted cycloalkyl, optionally substituted heterocyclyl, and optionally substituted heteroaryl. In some embodiments, R₁ is selected from optionally substituted cycloalkyl, or optionally substituted aryl. In some embodiments, R₁ is selected from optionally substituted phenyl and optionally substituted cyclohexyl. In some embodiments, R₁ is selected from phenyl and cyclohexyl.

In some embodiments, at least one of R₂ and R₃ is optionally substituted alkyl. In some embodiments, at least one of R₂ and R₃ is optionally substituted C₁-C₈alkyl. In some embodiments, at least one of R₂ and R₃ is C₁-C₅ alkyl. In some embodiments, the alkyl is methyl, ethyl, n-propyl or iso-propyl. In some embodiments, the alkyl is methyl.

The compound of Formula (I) may be selected from:

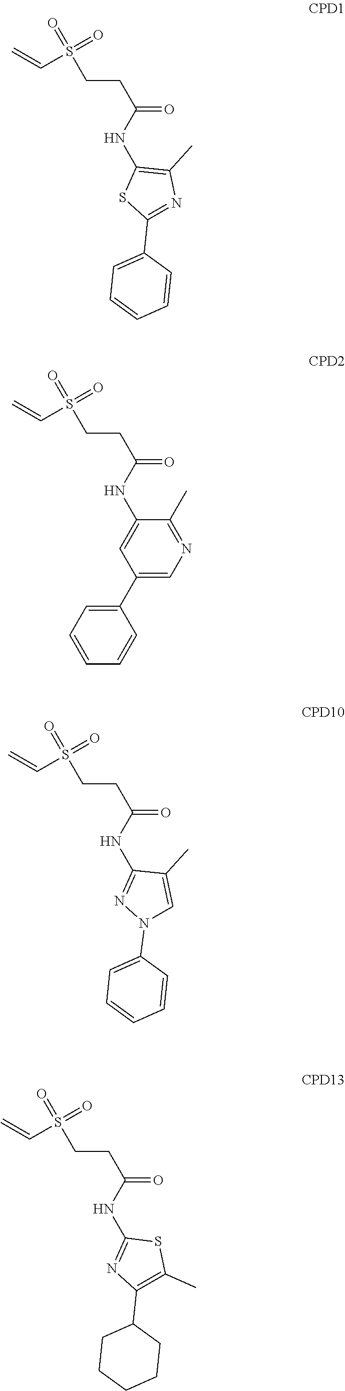

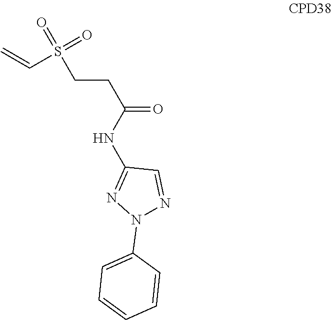 [SMILES: C=CS(=O)(=O)CCC(=O)Nc1cnn(-c2ccccc2)n1]

In some embodiments, compound of Formula (I) is selected from:

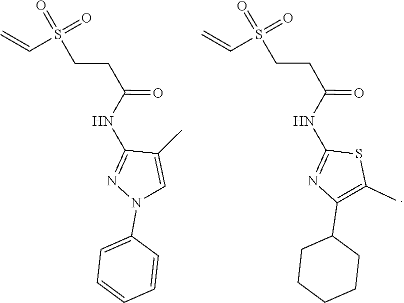 [SMILES: C=CS(=O)(=O)CCC(=O)Nc1nn(-c2ccccc2)cc1C ; C=CS(=O)(=O)CCC(=O)Nc1nc(C2CCCCC2)c(C)s1]

The present disclosure concerns a modulator of Hippo pathway, comprising a compound of Formula (I). The compound of Formula (I) is a modulator of YAP/TAZ-TEAD.

In some embodiments, the modulator is for use in vitro. For example, the modulators may be used to treat a cell line cell, or a tumour excised from an organism. In other embodiments, the modulator is for use in vivo.

The present disclosure also concerns a composition comprising a compound of Formula (I) or a salt, solvate or prodrug thereof, and palmitic acid or a salt, solvate or prodrug thereof.

The present disclosure also concerns a pharmaceutical composition comprising an effective amount of a compound of Formula (I) or a pharmaceutically acceptable salt, solvate or prodrug thereof, optionally in combination with a pharmaceutically acceptable carrier, excipient or diluent.

In some embodiments, the composition further comprises palmitic acid, or a pharmaceutically acceptable salt, solvate or prodrug thereof. For example, palmitate is the ionised form of palmitic acid, a fatty acid with a 16-carbon chain.

In some embodiments, the pharmaceutical composition further comprises another active ingredient. The active ingredient may be a cancer drug.

The compound of the invention can be administered to a subject as a pharmaceutically acceptable salt thereof. Suitable pharmaceutically acceptable salts include, but are not limited to salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium. In particular, the present invention includes within its scope cationic salts eg sodium or potassium salts, or alkyl esters (eg methyl, ethyl) of the phosphate group.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will be appreciated that any compound that is a prodrug of the compound of formula (I) is also within the scope and spirit of the invention. Thus the compound of the invention can be administered to a subject in the form of a pharmaceutically acceptable pro-drug. The term “pro-drug” is used in its broadest sense and encompasses those derivatives that are converted in vivo to the compound of the invention. Such derivatives would readily occur to those skilled in the art. Other texts which generally describe prodrugs (and the preparation thereof) include: Design of Prodrugs, 1985, H. Bundgaard (Elsevier); The Practice of Medicinal Chemistry, 1996, Camille G. Wermuth et al., Chapter 31 (Academic Press); and A Textbook of Drug Design and Development, 1991, Bundgaard et al., Chapter 5, (Harwood Academic Publishers).

The compound of the invention may be in crystalline form either as the free compound or as a solvate (e.g. hydrate) and it is intended that both forms are within the scope of the present invention. Methods of solvation are generally known within the art.

The compound of the invention, or a pharmaceutically acceptable salt, solvate or prodrug thereof is administered to the patient in a therapeutically effective amount. As used herein, a therapeutically effective amount is intended to include at least partially attaining the desired effect, or delaying the onset of, or inhibiting the progression of, or halting or reversing altogether the onset or progression of macular degeneration.

As used herein, the term “effective amount” relates to an amount of compound which, when administered according to a desired dosing regimen, provides the desired therapeutic activity. Dosing may occur at intervals of minutes, hours, days, weeks, months or years or continuously over any one of these periods. Suitable dosages may lie within the range of about 0.1 ng per kg of body weight to 1 g per kg of body weight per dosage, such as is in the range of 1 mg to 1 g per kg of body weight per dosage.

In one embodiment, the dosage may be in the range of 1 mg to 500 mg per kg of body weight per dosage. In another embodiment, the dosage may be in the range of 1 mg to 250 mg per kg of body weight per dosage. In yet another embodiment, the dosage may be in the range of 1 mg to 100 mg per kg of body weight per dosage, such as up to 50 mg per body weight per dosage.

Suitable dosage amounts and dosing regimens can be determined by the attending physician and may depend on the severity of the condition as well as the general age, health and weight of the patient to be treated.

The compound of the invention may be administered in a single dose or a series of doses. While it is possible for the active ingredient to be administered alone, it is preferable to present it as a composition, preferably as a pharmaceutical composition. The formulation of such compositions is well known to those skilled in the art. The composition may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The carrier must be pharmaceutically “acceptable” in the sense of being compatible with the other ingredients of the composition and not injurious to the patient. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Injectables for such use can be prepared in conventional forms, either as a liquid solution or suspension or in a solid form suitable for preparation as a solution or suspension in a liquid prior to injection, or as an emulsion. Carriers can include, for example, water, saline (e.g., normal saline (NS), phosphate-buffered saline (PBS), balanced saline solution (BSS)), sodium lactate Ringer's solution, dextrose, glycerol, ethanol, and the like; and if desired, minor amounts of auxiliary substances, such as wetting or emulsifying agents, buffers, and the like can be added. Proper fluidity can be maintained, for example, by using a coating such as lecithin, by maintaining the required particle size in the case of dispersion and by using surfactants. By way of example, the compound, composition or combination can be dissolved in a pharmaceutically effective carrier and be injected into the vitreous of the eye with a fine gauge hollow bore needle (e.g., 30 gauge, ½ or ⅜ inch needle) using a temporal approach (e.g., about 3 to about 4 mm posterior to the limbus for human eye to avoid damaging the lens).

A person skilled in the art will appreciate that other means for injecting and/or administering the compound, composition or combinations to the vitreous of the eye can also be used. These other means can include, for example, intravitreal medical delivery devices. These devices and methods can include, for example, intravitreal medicine delivery devices, and biodegradable polymer delivery members that are inserted in the eye for long term delivery of medicaments. These devices and methods can further include transscleral delivery devices.

Other modes of administration including topical or intravenous administration may also be possible. For example, solutions or suspensions of the compound, composition or combinations of the invention may be formulated as eye drops, or as a membranous ocular patch, which is applied directly to the surface of the eye. Topical application typically involves administering the compound of the invention in an amount between 0.1 ng and 10 mg.

The compound, composition or combinations of the invention may also be suitable for intravenous administration. For example, a compound of formula (I) or a pharmaceutically acceptable salt, solvate or prodrug thereof may be administered intravenously at a dose of up to 16 mg/m².

The compound, composition or combinations of the invention may also be suitable for oral administration and may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste. In another embodiment, the compound of formula (I) or a pharmaceutically acceptable salt, solvate or prodrug is orally administrable.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g inert diluent, preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

The compound, composition or combinations of the invention may be suitable for topical administration in the mouth including lozenges comprising the active ingredient in a flavoured base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

The compound, composition or combinations of the invention may be suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the compounds of the invention.

The compound, composition or combination of the invention may be suitable for parenteral administration include aqueous and non-aqueous isotonic sterile injection solutions which may contain anti-oxidants, buffers, bactericides and solutes which render the compound, composition or combination isotonic with the blood of the intended recipient; and aqueous and non-aqueous sterile suspensions which may include suspending agents and thickening agents. The compound, composition or combination may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage composition or combinations are those containing a daily dose or unit, daily sub-dose, as herein above described, or an appropriate fraction thereof, of the active ingredient.

It should be understood that in addition to the active ingredients particularly mentioned above, the composition or combination of this invention may include other agents conventional in the art having regard to the type of composition or combination in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

The present disclosure also concerns a method of treating cancer in a patient in need thereof, comprising administering to the patient a therapeutically effective amount of a compound of Formula (I) or a pharmaceutically acceptable salt, solvate or prodrug thereof.

The present disclosure also concerns a compound of Formula (I) or a pharmaceutically acceptable salt, solvate or prodrug thereof for use in the treatment of cancer.

The present disclosure also concerns a use of a compound of Formula (I) or a pharmaceutically acceptable salt, solvate or prodrug thereof in the manufacture of a medicament for treating cancer in patient in need thereof.

In some embodiments, the cancer is characterised by an elevated YAP/TAZ-TEAD activity and/or drug resistance.

In some embodiments, the cancer is selected from mesothelioma, liver cancer, gastric cancer, metastatic non-small-cell lung cancer (NSCLC) and colorectal cancer.

In some embodiments, the method, compound or use thereof further comprises another active ingredient. The active ingredient may be a cancer drug.

In some embodiments, the method comprises administering to the patient a therapeutically effective amount of a compound of Formula (I) or a pharmaceutically acceptable salt, solvate or prodrug thereof in combination with an effective amount of a cancer active ingredient.

In some embodiments, the compound of Formula (I) or a pharmaceutically acceptable salt, solvate or prodrug thereof is used in combination with a cancer active ingredient for use in the treatment of cancer.

In an embodiment, the present disclosure provides a use of a compound of formula (I) or a salt, solvate or prodrug thereof in the manufacture of a medicament for treating cancer in combination with a cancer active ingredient.

In an embodiment, the present disclosure provides a use of a cancer active ingredient in the manufacture of a medicament for treating cancer in combination with the compound of formula (I) or a salt, solvate or prodrug thereof.

In some embodiments, the compound of formula (I) or a salt, solvate or prodrug thereof and the cancer active ingredient are added simultaneously or sequentially in any order.

As used herein, the term “combination” relates to the co-administration of the combination partners to a single patient, and are intended to include treatment regimens in which the agents are not necessarily administered by the same route of administration or at the same time. The therapeutic compounds or treatments used in such combination therapies may be administered together with a compound of formula (I) or a pharmaceutically acceptable salt, solvate or prodrug, one after the other, separately in one combined unit dosage or in separate unit dosage forms.

### Examples

FIG. 3C shows MS/MS results of the compounds reaction with TEAD, which revealed that conserved cysteine is modified in vitro. It is observed that:
Four cysteine residues per TEAD-YBD
Four peptides, each containing one cysteine
% modified peptide=number of modified peptide/total peptide detected
Cysteine known to be palmitoylated was preferentially modified by compounds
SPLCEYMINFIHK peptide containing the known conserved cysteine is a major target
VCSFGK peptide containing the other cysteine known to palmitoylated was revealed

FIG. 4 shows CPD10 and CPD13 inhibits the expression of YAP/TAZ proteins. Method: A549 cells (at 60% confluency) either mock treated or treated with indicated doses of CPD10 and CPD13 for 24 hrs. Cells were washed with ice-cold 1×PBS and lysed in protein lysis buffer. Whole cell lysates were quantified by Pierce™ BCA Protein assay kit (Catalog number: 23225) and denatured in 4×LDS buffer (Thermo catalog number: NP0007) added with competing amount of DTT followed by boiling for 3 minutes. Samples were resolved in 8% of SDS-PAGE, transferred onto nitrocellulose membrane and immunoblotting was performed with indicated antibodies.

GFP-TRAP assay: To purify TEAD4 binders, GFP-Trap Magnetic Agarose (gtma-100) was used, and assay was performed strictly according to manufacturer's instructions. At the end of the assay, agarose beads were resuspended in 2×LDS lysis buffer, competing amount of DTT was added into it and samples were boiled for 3 minutes. Supernatants were collected and used for immunoblotting.

Result: To determine the CPD10 and CPD13 mediated expression of hippo signaling target proteins in A549 cells, whole cell lysates from mock treated and compounds treated cells were immunoblotted to determine the level of TEAD4, Pan-TEADs, YAP, TAZ proteins. Interestingly, in response to CPD10 treatment, YAP expression was downregulated in a dose dependent manner. TAZ expression was significantly reduced at 10 Um. At the highest concentration, TEAD4 and pan-TEAD expression was also reduced (A). Similar to CPD10, CPD13 downregulate YAP expression at 10 Um (B). To determine the level of other transcriptional targets, we tested the p53 protein in same sets of protein samples. Compound 10 and 13 didn't resulted in change in the expression of wild type p53 expression, suggesting that this compound specifically acting in hippo signaling pathway. GAPDH was used as loading control.

To determine whether CPD10 or 13 leads downregulation of TEAD4 binding to its transcriptional targets YAP and TAZ, U20S cells stably expressing GFP alone and GFP fused TEAD4 were either mock-treated or treated with indicated doses of CPD10. GFP-TRAP assay was performed to pull down TEAD4 binders, and immunoblotted. As one of the strong binders of the TEAD4, YAP/TAZ protein signal were detected in purified complex. Interestingly, this binding was significantly reduced in response to 10 uM of CPD10 treatment. The GFP blot indicating the GFP and GFP fused TEAD4 signal in whole cell lysates and purified protein samples (C).

FIG. 5 shows CPD10 and CPD13 treatment resulted in TEAD4 cytoplasmic localization. Methods: U20S cells stably expressing GFP-TEAD4 were seeded at 50,000 cell densities onto glass cover slips in six well plates. Next day, cells were either DMSO treated or treated with indicated compounds at 10 μM concentrations. After 18 hrs of treatment cells were washed and fixed in 4% formaldehyde for 20 minutes. Blocking was done in 3% BSA dissolved in PBST for 1 hour. Cell were incubated with indicated antibodies for overnight in a cold room. Next day, after washing 3× in PBST samples were incubated with respective secondary antibodies for 45 minutes at room temperature. After washing nuclear staining was done with DAPI for 10 minutes. Coverslips were added onto glass slides with the help of mounting medium. After drying, cells were imaged at 63× objective and processed with ZEN x64 blue software.

Result: In DMSO treated control samples we observed very nuclear localized TEAD4 (green) signal, strongly imposed to the DAPI signal. Nuclear and cytoplasmic YAP and TAZ was observed either in DMSO or in compound treated cells. Interestingly, in responses to CPD10 and 13 treatment we detected GFP signal from outside the nucleus. While CPD10 or CD13 treatment didn't diminish the whole nuclear TEAD4 presence, these two inhibitors lead to induction of cytoplasmic localization of the TEAD4 protein.

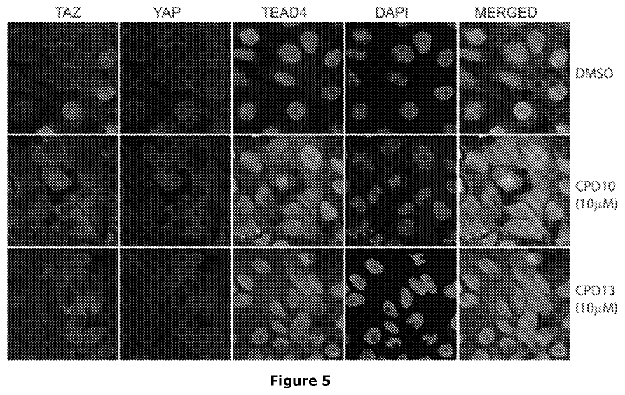

FIG. 6 shows CPD10 and CPD13 dependent Transcriptional target gene regulation. Method: A549 cells (at 60% confluency) either mock treated or treated with indicated doses of CPD 10 and CPD13 for 24 hrs. Cells were washed with ice-cold 1×PBS. RNA extraction was performed with Quick-RNA™ miniprep kit (Zymo Research, Cat. No. R1055), strictly according to manufacturer's instructions. Nanodrop quantified 2 μg of RNA samples were used for cDNA synthesis with cDNA Reverse Transcription Kit (Thermo Cat. No. 4368814) strictly according to the manufacturer's instructions. In RT PCR experiment, comparative target gene expression was performed by SYBR™ Green (Thermo Cat. No. 4309155) Master mix. Relative target gene expressions were compared and plotted on Y-Axis.

Result: Relative expression of YAP and TAZ in response to low dose of CPD10 (A) and CPD13 (B) treatment in A549 cells. c-MYC was included as non-transcriptional target of the TEADs. NFKB1 was included as its expression is reciprocal to the YAP/TAZ. Downstream effectors of the TEADs targets (CYR61, AXL, CTGF and ANKRD1) were determined in responses to 2.5, 5 and 10 μM of CPD10 (C) and CPD13 (D).

CPD10 and CPD13 Downregulate the TEADs transcriptional target genes: To determine CPD10 and CPD13 induced relative expression of TEADs target genes, A549 cells were treated with indicated doses for 24 hrs. Transcriptional profiling was performed with respective primer pairs. In response to 2.5 μM of CPD10, YAP/TAZ expression was downregulated. A minor increase in NFKB1 was observed. Further we tested the expression of one of the strong non hippo transcription factor, the C-Myc. As expected, c-MYC expression was not affected in response to CPD10 treatment (A). In response to CPD13 treatment, a significant reduction of YAP/TAZ expression was detected. C-Myc expression was not affected. Earlier findings suggest that YAP attenuates NF-κB pathway. An Increase of NFKB1 expression is consistent with significant downregulation of YAP expression in response to CPD13 treatment (B). To determine the state of TEADs transcriptional target genes at higher concentrations, A549 cells were treated with indicated doses of compounds and analyzed for the expression of CYR61, AXL, CTGF and ANKRD1. Interestingly in response to CPD10, majority of targets (CYR61, AXL and CTGF) suppressed at 2.5 μM and subsequently downregulated in a dose dependent manner. The expression of ANKRD1 was significantly reduced at 10 μM (C). A549 cells treated with indicated dose of CPD13 resulted in significant reduction on all four test targets (CYR61, AXL, CTGF and ANKRD1) at 2.5 um, which was further downregulated in a dose dependent manner (D). Overall, these data suggest TEAD4 inhibition by CPD10 and CPD13 resulted in downregulation of Hippo signaling target genes.

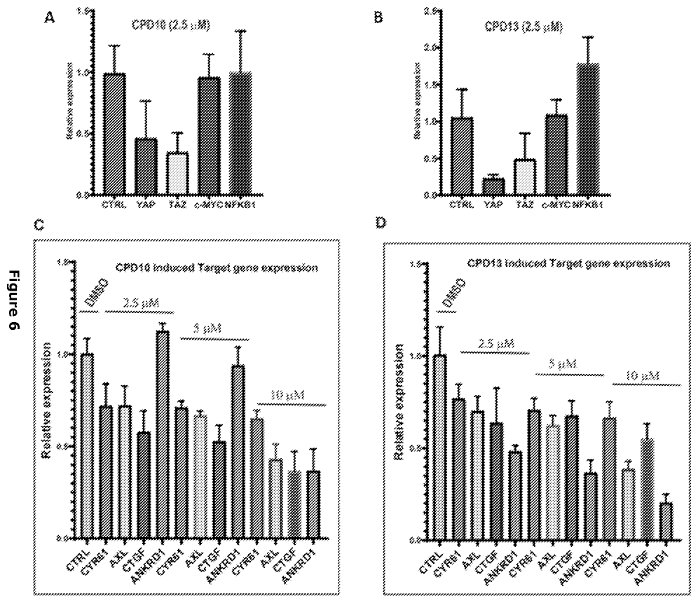

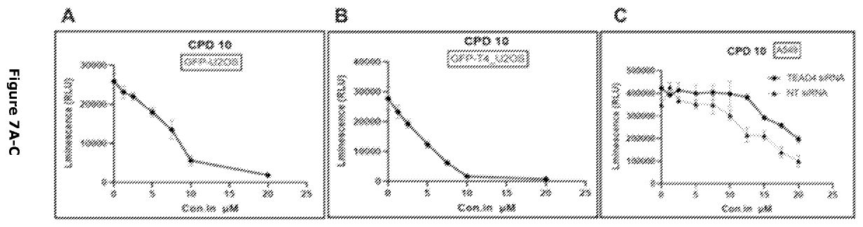

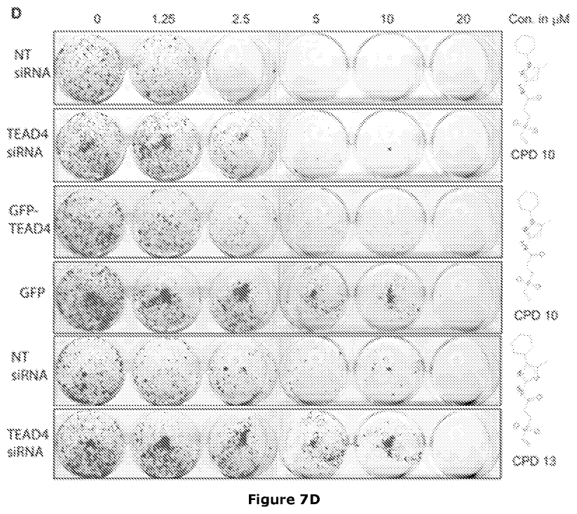

FIG. 7 shows Cell Proliferation and Colony formation assay.

Method: Indicated cells were plated at 3000 cells/80 μl in each well. TEAD4 depletion was performed by reverse transfection with 20 nM of Dharmacon smart pool NTsiRNA and TEAD4 siRNA in 0.25 ul of lipofectamine mixed with 25% of OPTIMEM. After 18 hrs of transfection, cells were recovered for 24 hrs in fresh DMEM medium. Cells were treated with indicated compounds at different doses. After 48 hrs of dosing cells were recovered in fresh medium for 3 days. Cell Titre Glow (CTG) assay was used.

In colony forming assay, indicated cells were plated at 5,000 cells density in 2 ml of DMEM medium. TEAD4 depletion was performed by reverse transfection with 20 nM of Dharmacon smart pool NTsiRNA and TEAD4 siRNA in 2.5 ul of lipofectamine mixed with 25% of OPTIMEM. After 18 hrs of transfection, cells were either DMSO treated or treated with indicated compounds at different doses. After 48 hrs of the treatment cells were recovered in a fresh medium for 4 days. At the end of the experiment cells were Glutaraldehyde fixed and stained in crystal violet solution for 2 hrs. After washing, plates were scanned for image processing.

Results: Level of CPD10 and CPD13 displayed TEAD4 dependent cellular proliferation and differential sensitivity in U2OS and A549 cells. U20S cells stably expressing GFP fused TEAD4 and GFP alone were treated with different doses of CPD10 and analysed for cellular proliferation. The U20S cells expressing GFP alone didn't display sensitivity at low dose of the CPD10 treatment (A), while overexpressing GFP-TEAD4 displayed higher sensitivity below 5 uM of CPD10 treatment (B). Further we used cell lines expressing higher level of TEAD4 and performed depletion experiment followed by comparative sensitivity analysis. Interestingly, unlike NTsi treated cells, TEAD4 depleted A549 cells displayed lower sensitivity indicating that CPD10 activity is TEAD4 dependent (C).

In line with proliferation assay we performed colony forming assay in U20S cells either depleted or overexpressing TEAD4. Cells stably expressing GFP and GFP fused TEAD4 were also included in this assay. Unlike TEAD4 depleted cells, U20S cells treated with control siRNA displayed very high sensitivity at 2.5 μM of CPD10 treatment. Similarly, U20S cells overexpressing GFP fused TEAD4 displayed very high sensitivity in comparison to GFP alone expressing cells. We also tested our CPD13 in siRNA depletion experiment and we observed that like CPD10, CPD13 dependent cellular sensitivity is TEAD4 dependent (C, lower panels). Taken together these data suggest that CPD10 and CPD13 mediated cellular proliferation and sensitivity is TEAD4 dependent.

FIG. 8 shows TEAD modelling and evaluation of CPD10 and CPD13 in NCI-H226 mesothelioma cell line.

Method: Modeling TEAD-CPD10/CPD13 using in-silico docking.

Evaluation of CPD10 and CPD13 in NCI-H226 mesothelioma cell line: Actively growing cells were either DMSO added or treated with indicated concentrations of CPD10 and CPD13 for 24 hrs. Cells were collected in ice-cold PBS and pellets were lysed in IP Lysis Buffer (25 mM Tris-HCl pH 7.4, 150 mM NaCl, 1 mM EDTA, 1% NP-40 and 5% glycerol and competing amount of protease inhibitor). Samples were lysed by sonication, BCA assay was performed and equal concentration of protein were added with TEAD4 antibody (3 ul). After overnight incubation in cold room, samples were added with anti-rabbit magnetic beads to trap the TEAD and binding complex for 2 hrs. Samples were washed 3 times and bound fraction were collected with the help of magnetic stand. Samples were added with 4×LDS, competing amount of DTT, and boiled for 3 minutes. Whole cell lysate (Input) and IP samples were resolved in 8% SDS-PAGE gel and immunoblotted with indicated antibodies.

Relative quantification of immunoblot signals were quantified with IMAGE J software. Actively growing cells were added with indicated concentrations of CPD10 and CPD13 and cells were collected after 24 hrs. RNA was extracted and cDNA was synthesized. Indicated primer pairs were used for RT-PCR. Relative gene expression was determined by using GAPDH amplification.

Colony forming assay was essentially performed as described in previous section.

### Result:

Vinyl sulfone warhead forms covalent bond with Cys359. Sulfone forms h-bond interactions with side chain of Lysine (Lys336) and backbone of Cystine (Cys359). Rest of the molecule occupies the hydrophobic pocket.

Endogenous TEAD4 IP indicate a strong binding between YAP-TEAD, which was disrupted to a great extent in CPD10 and CPD13 treated samples. The signal intensity quantification data indicate a significant reduction in YAP-TEAD binding. Transcriptional profiling data indicate that CPD10 and CPD13 leads to repression of TEAD transcriptional target genes. Colony forming assay data indicating that CPD10 and CPD13 is strongly sensitizes NCI-H226 mesothelioma cells which demonstrates their therapeutic potential.

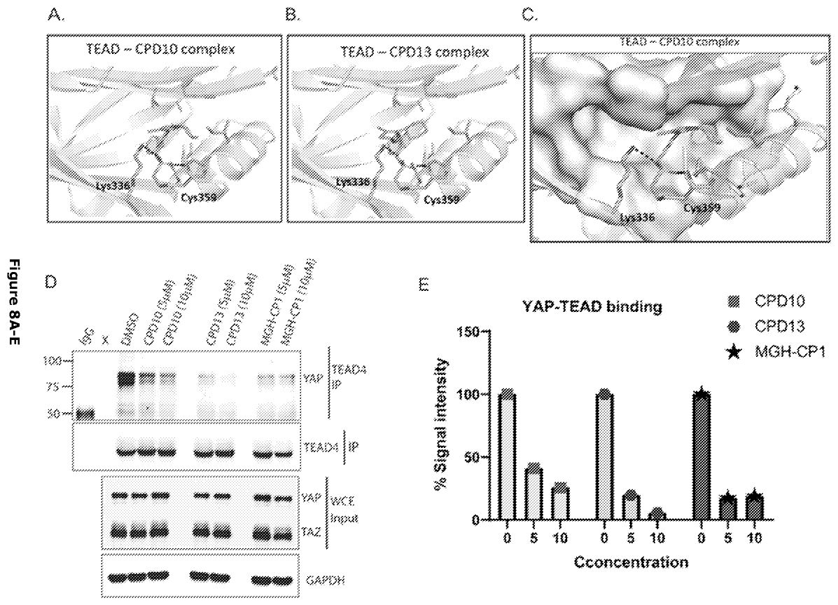

FIG. 9 shows the down regulation of Hippo-dependent gene transcripts. RT-qPCR revealed dose dependent decrease of CTGF and CYR61 transcripts. MGH-CP1 (positive control) and Cpd 13 were tested on MCF10 and A549.

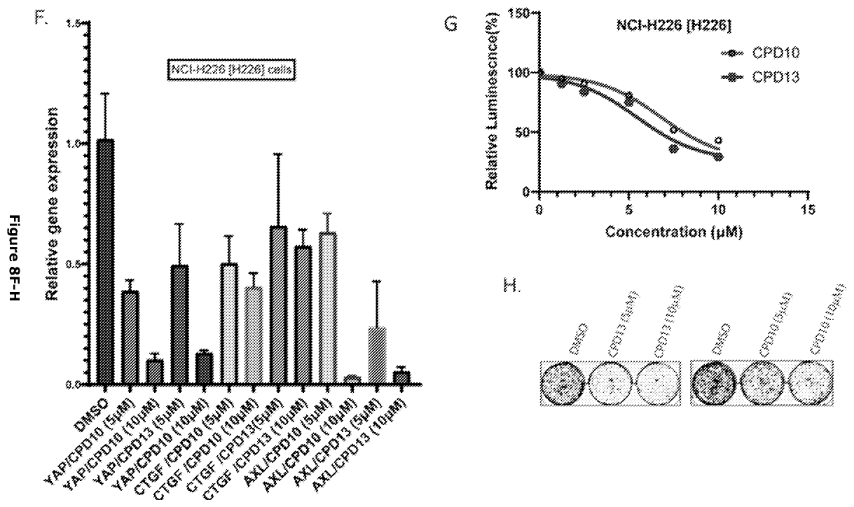

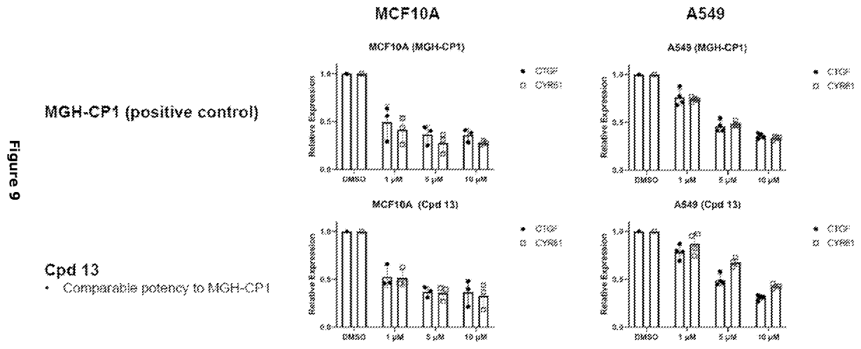

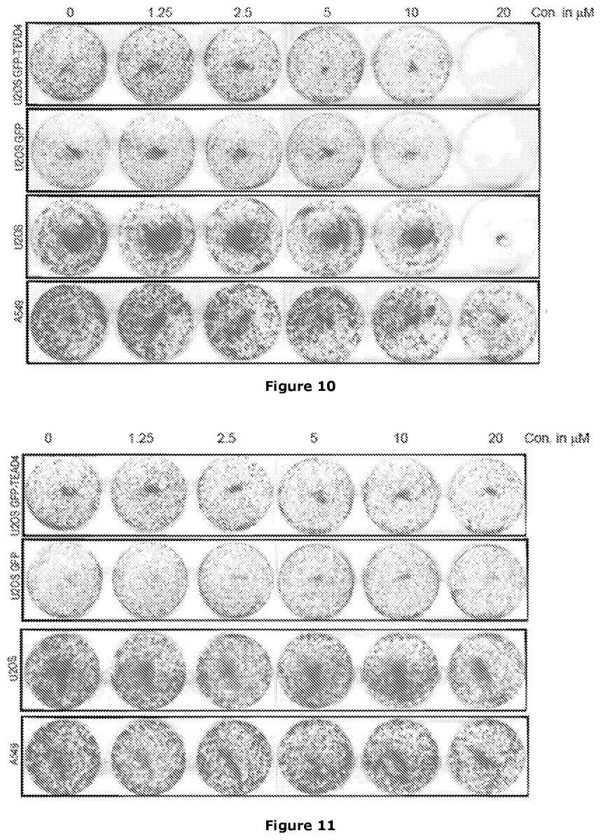

Colony forming assay data for compound 2 and 38 are shown in FIGS. 10 and 11 respectively. The results suggest that CPD2 and CPD38 induced cell sensitivity is not TEAD dependent.

The above shows that TEAD is an attractive target for cancer therapeutics.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A compound of Formula (I) or a salt, solvate or prodrug thereof: 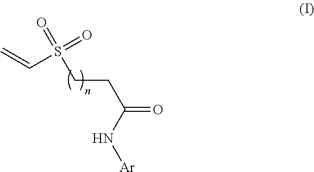
wherein
n is an integer selected from 1 to 5; and
Ar is an optionally substituted aryl or optionally substituted heteroaryl.

2. The compound according to claim 1, wherein Ar is optionally substituted heteroaryl.

3. The compound according to claim 1, wherein Ar is an optionally substituted heteroaryl, wherein a heteroatom is at a 2′ position relative to N of the amide moiety.

4. The compound according to claim 1, wherein Ar is selected from optionally substituted phenyl, optionally substituted pyridinyl, optionally substituted thiazolyl, optionally substituted pyrazolyl, or optionally substituted triazolyl.

5. The compound according to claim 1, wherein n is an integer selected from 1 to 4.

6. The compound according to claim 1, wherein the compound of Formula (I) is a compound of Formula (Ia): 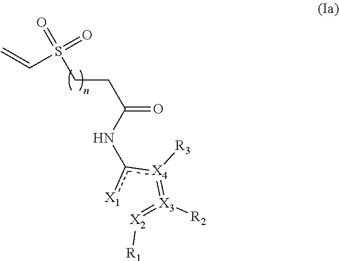
wherein
n is an integer selected from 1 to 5;
X₁ is a heteroatom selected from N, S, or O;
X₂, X₃ and X₄ are independently selected from C, N, O, or S;
when X₂ is N or C, R₁ is selected from optionally substituted cycloalkyl, optionally substituted heterocyclyl, optionally substituted aryl, or optionally substituted heteroaryls;
when X₃ and X₄ are independently N or C, R₂ and R₃ are independently selected from H, or optionally substituted alkyl.

7. The compound according to claim 6, wherein X₁ is a heteroatom selected from N, or S.

8. The compound according to claim 6, wherein at least one of X₂, X₃ and X₄ is N, O, or S.

9. The compound according to claim 6, wherein R₁ is selected from optionally substituted cycloalkyl, or optionally substituted aryl.

10. The compound according to claim 6, wherein at least one of R₂ and R₃ is optionally substituted alkyl.

11. The compound according to claim 1, wherein the compound of Formula (I) is selected from:

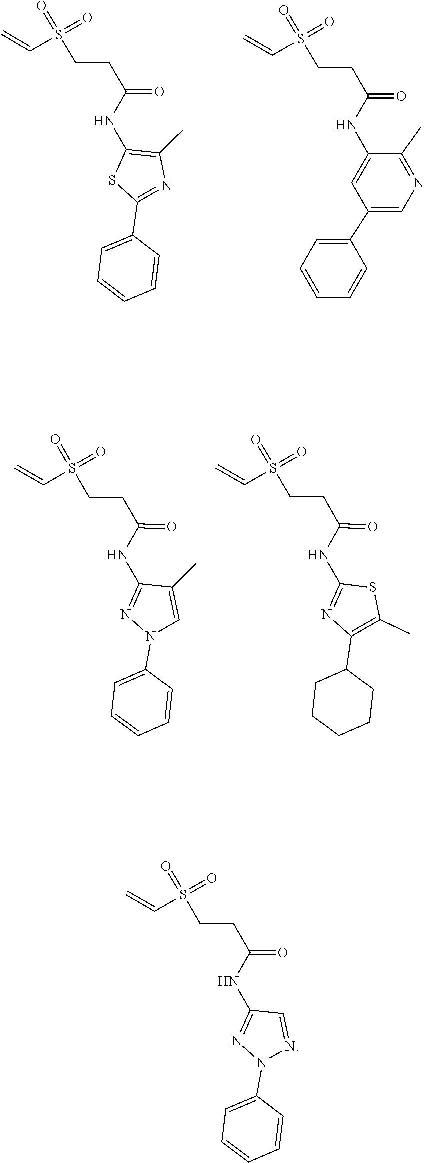 [SMILES: C=CS(=O)(=O)CCC(=O)Nc1sc(-c2ccccc2)nc1C ; C=CS(=O)(=O)CCC(=O)Nc1cc(-c2ccccc2)cnc1C ; C=CS(=O)(=O)CCC(=O)Nc1nn(-c2ccccc2)cc1C ; C=CS(=O)(=O)CCC(=O)Nc1nc(C2CCCCC2)c(C)s1 ; C=CS(=O)(=O)CCC(=O)Nc1cnn(-c2ccccc2)n1]

12. A modulator of Hippo pathway, wherein the modulator is a compound of Formula (I) according to claim 1.

13. A pharmaceutical composition comprising an effective amount of a compound of Formula (I) according to claim 1 or a pharmaceutically acceptable salt, solvate or prodrug thereof, optionally in combination with a pharmaceutically acceptable carrier, excipient or diluent.

14. The pharmaceutical composition according to claim 13, wherein the pharmaceutical composition further comprises an active ingredient.

15. A method of treating cancer in a patient in need thereof, comprising administering to the patient a therapeutically effective amount of a compound of Formula (I) according to claim 1 or a pharmaceutically acceptable salt, solvate or prodrug thereof.

16.-17. (canceled)

18. The method of claim 15, wherein the cancer is characterised by an elevated YAP/TAZ-TEAD activity and/or drug resistance.

19. The method of claim 15, wherein the cancer is selected from mesothelioma, liver cancer, gastric cancer, metastatic non-small-cell lung cancer (NSCLC) and colorectal cancer.

20. The method of claim 15, wherein the compound of Formula (I) is used in combination with an active ingredient.
